(19) United States
(12) Patent Application Publication
(10) Pub. No.: US 2012/0122224 A1
(43) Pub. Date: May 17, 2012

(54) DEVICE FOR PERFORMING PHOTOCHEMICAL PROCESSES

(75) Inventors: Frank Schael, Darmstadt (DE); Karoly Nagy, Aachen (DE)

(73) Assignee: EHRFELD MIKROTECHNIK BTS GMBH, Wendelsheim (DE)

(21) Appl. No.: 13/319,136

(22) PCT Filed: May 5, 2010
(86) PCT No.: PCT/EP10/02748

§ 371 Date: Jan. 23, 2012

(30) Foreign Application Priority Data
May 8, 2009 (DE) 10 2009 020 527.6

(51) Int. Cl. C12N 5/04; C12N 1/20; C12M 1/42; C12M 3/00

(52) U.S. Cl. 435/420; 435/292.1; 435/252.1

(57) ABSTRACT

Device for carrying out photochemical processes on a microscale and use of the device for photochemical reactions and culturing photosynthesizing cells and/or microorganisms.

The present invention relates to a device for carrying out photochemical processes on a microscale, and also the use of the device according to the invention for culturing photosynthesizing cells and/or microorganisms.

Photochemical reactions are used, inter alia, in the industrial synthesis of chemical compounds, e.g. in the fields of pharmaceuticals, plant protection agents, aroma substances and vitamins. The expression photochemical reactions is taken to mean reactions which are initiated and/or maintained by electromagnetic radiation preferably in the UV range to the visible range.

Photobiotechnological processes play a role in the culturing of plant cells and plants, but also of photosynthesizing bacteria. By means of the radiation by electromagnetic radiation from artificial sources or in the form of sunlight, cells or microorganisms which photosynthesize are cultured.

Culturing is taken to mean the provision and maintenance of conditions which ensure growth and multiplication of the cells and/or microorganisms.

Photochemical and/or photobiotechnological processes also take place in the inactivation of microorganisms and viruses by UV radiation.

In the said processes which are termed hereinafter for short as photochemical processes, there is a challenge to ensure a uniform radiation of a medium.

The performance of technical apparatuses for carrying out photochemical processes is frequently limited by the depth of penetration of the electromagnetic radiation into the medium that is to be radiated. In particular in the case of biological media (e.g. algal cultures, blood, milk) that have high opacity, the depth of penetration of electromagnetic radiation is frequently restricted to a range of a few micrometres below the media surface.

In addition, it is of importance to avoid shadowing effects in the irradiation chamber. Shadowing effects lead to either good mixing needing to be established in the apparatuses in the irradiation zone and/or the medium needing to be circulated until a desired degree of conversion is achieved.

The risk of non-uniform irradiation is very high in this case. There is the risk that parts of the medium receive an excessive radiation dose and other parts of the medium receive an insufficient radiation dose. In the inactivation of microorganisms and/or viruses in biological products such as, for example, foods or blood plasma, there is the danger, e.g., that the parts which undergo an excessive radiation dose become irreversibly damaged, whereas in the parts which receive an insufficient radiation dose there is incomplete inactivation of viruses and/or microorganisms.

Photochemical processes, therefore, are frequently carried out in falling-film reactors in industry in order to utilize the formation of a film having low optical layer thicknesses for maximizing the irradiated volume and for minimizing shadowing effects of the incident radiation. These reactors have the disadvantage that the layer thickness is generally a function of the operating conditions such as flow rate and temperature and of material properties such as viscosity, and virtually cannot be adjusted independently.

DE 102 22 214 A1 describes, for example, a photobioreactor in which a plurality of compartments which are separated by light-permeable walls and have layer thicknesses of 5 to 30 mm are formed in order to permit improved utilization of light and parallel operation at high to low light intensities. Since the optimum light utilization depends on the light absorption of the cell suspension and therefore on the cell count, the efficiency of such a device varies during the growth phase of the cells.

GB 2118572 describes an apparatus for carrying out a photobioreaction with a liquid cell culture. The apparatus consists of a light-permeable part in which the medium is conducted in a turbulent manner and a top reservoir. For transport there serves a peristaltic pump, or a pressure difference generated by gas input. The system contains, for example, 4.6 1 of medium, wherein about 41 flow through the part that can be irradiated. The gas discharge of inhibiting oxygen and also the input of CO2 proceeds in the direction of flow downstream of the reactor in riser pipes in which a separation of gas/liquid phase is carried out.

The setup is for photochemical and photobiological processes in which a relatively rapid gas consumption does not proceed optimally, since here no gas can be resupplied to the solution from the gas phase within the irradiation zone. In addition, the discharge of gases formed in the irradiation zone, as in photobiological processes, for example oxygen, does not proceed at the site of gas generation, and so a growth-inhibiting activity of the oxygen in the reactor cannot be excluded. The design in which the medium in the setup is transported by pure gas addition is not ideal, since, because of a change in media properties during the growth of the microorganisms, media properties such as viscosity are changed and therefore the liquid velocity cannot be kept constant over the growth phases, or only with particular effort. Owing to the foam formation which is unavoidable in this mode of operation, in the publication, antifoam is also used, which is not desirable for all applications.

The microprocessing technique or microreaction technique has in recent years increasingly become an important tool in chemistry and in research and development. The cause is the demand of the market to develop novel products and improved processes in increasingly short times.

The modular microprocessing technique offers the possibility of combining various microprocess modules in the manner of building blocks to form a complete production system in a very small format. In addition to the resultant high flexibility and reduction of wastes owing to the decreased amounts of chemicals that are required for experiments in microreaction systems, the microprocessing technique has direct advantages for chemical process engineering: microstructured apparatuses have a very high ratio of surface area to volume. For this reason, for example heat and mass transport operations may be markedly intensified.

The high ratio of surface areas to volume can also be utilized for markedly improving the radiation transport in a reaction solution compared with conventional photochemical apparatuses. The ratios in the conventional plants for photochemical reactions frequently lead, for example, to only small concentrations of starting materials being able to be used. This is in part the consequence of the fact that the thickness of the irradiated liquid layer cannot be readily controlled.

Conversely, the small characteristic dimensions of microstructured apparatuses in the range of typically 1 to 2000 μm, in addition to the particular advantages, also give rise to particular challenges which, in comparison with the macroscale plant engineering, require technically different solutions, in particular the use of matched multifunctional systems (see, for example, V. Hessel, S. Hardt, H. Lowe, “Chemical Micro Processing Engineering”, vol. 1-2, Wiley-VCH, Weinheim, 2005).

Small dimensions generally bring, for example, high shear stresses for the media that are to be irradiated, as a result of which culturing microorganisms is made difficult and possible only taking into account a multiplicity of interrelated boundary conditions. The combination of various characteristics of the apparatuses is subject matter of experimental studies and is very difficult to predict.

In H. Ehrich et al., Application of Microstructured Reactor Technology for the Photochemical Chlorination of Alkylaromatics, Chimia 56 (2002), pp. 647 to 653, the use of a microfalling-film reactor for selective photochlorination of toluene 2,4-diisocyanate is described. A corresponding microfalling-film reactor is also described in DE10162801A1. Although this reactor permits by means of a window radiation to be coupled in, it does not utilize the complete amount of incident radiation, since some is shadowed due to the construction. In addition, this reactor has the disadvantage that the residence time and irradiation time cannot be controlled over a broad range, because in the falling-film principle there is always the risk that the film tears off

Commercially available setups for culturing photosynthesizing cells and microorganisms are based either on rectangular glass vessels which are not recirculated by pumping, or irradiated tube coils with equilibration vessels in which sensors are accommodated and mass transfer proceeds. Here the available optical layer thicknesses are in the centimetre range. DE29607285U1 describes, for example, a photobioreactor having a plate-shaped appliance for culturing photosynthesizing microorganisms. The exchange of gases here is only possible in the intermediate vessel.

DE4411486C1 describes a method for culturing and fermenting microorganisms using an ultrathin film-like media stream between matter- and light-permeable material, e.g. made of PE films. Carbon dioxide and oxygen exchange proceeds through the films. The thin layer having 50 to 500 times the diameter of the cells is distributed, e.g., via oscillating sprinklers on to the gap and then flows through the gap under the force of gravity. Owing to the passage of matter through a film, the exchange rate is limited. The light source used is a Na vapour lamp. The circulation rate of the medium results from the flow velocity of the medium through the gap and is not actively fixed by a transport element.

C.-G. Lee and B. O. Palsson (High-density algal photobioreactors using light-emitting diodes, Biotechnology and Bioengineering, 44, 1161-1167 (1994)) describe a setup for algal growth using LED illumination in a rectangular glass vessel through which flow passes and media treatment by ultrafiltration. Here it is reported, in particular, that when the vessel thickness is decreased the cell count density of the culture can be increased. The reason for this is the better utilization of radiation and decreased shadowing effects. By means of the combination of the radiator with a rectangular vessel without further fluid guidance, however, the reactor surface cannot be optimally illuminated by the spot-like emission characteristics.

Whereas for culturing algae, a number of setups are known and in part tested in large-scale experiments, there remains a further need for improvement with respect to a number of technical properties such as utilization and type of the radiation sources, gas input, pH stabilization, control and monitoring of culture conditions, finally with the purpose of being able to operate the plants more economically.

Proceeding from the prior art, the object is therefore to provide a device for carrying out photochemical processes which has a high degree of efficiency with respect to the irradiated electromagnetic radiation. The sought-after device must at the same time ensure a well-defined and uniform irradiation of a medium.

In particular, the radiation dose which the irradiated medium experiences must be adjustable. The sought-after device needs to be operated either continuously or in batch operation as well. Where possible it needs to be made up in a modular manner and be flexible in use thereby. It needs to be simple in handling and inexpensive. It needs to enable photochemical processes to be carried out under economic conditions.

According to the invention this object is achieved by a microstructured device according to claim 1. The present invention therefore relates to a device for carrying out photochemical processes, comprising at least an irradiation zone, a source of electromagnetic radiation and means for transporting a medium through the device, characterized in that the volume of the irradiation zone corresponds to at least 0.5 times the device volume.

The device volume is taken to mean the volume of the device which is composed of the volumes of the irradiation zone, the feed lines and outlet lines, the means for transporting the medium and any further components of the device. Storage vessels from which media are fed into the device according to the invention and also collection vessels for receiving products are not included in the device volume in this case.

A device according to the invention having an irradiated volume which is at least as large as the unirradiated volume of the device has the advantage that the radiation energy can be utilized very efficiently.

Suitable sources of electromagnetic radiation are all radiation sources known to those skilled in the art that emit electromagnetic radiation of the desired wavelength or of the desired wavelength range. Preferably, one or more light-emitting diodes are used.

A light-emitting diode is an electronic semiconductor component in which the emission of electromagnetic radiation can be excited by a flow of current in the forward direction of the diode. The emitted wavelengths are dependent on the semiconductor material used. Light-emitting diodes, compared with, e.g., incandescent lamps, have the advantage that they are not thermal radiators and therefore do not unnecessarily heat the medium or parts of the device. They emit light in a limited spectral range; the light is virtually monochrome. The efficiency is high and light-emitting diodes therefore permit targeted and efficient use of the emitted photons.

Preferably, in the device according to the invention, a plurality of light-emitting diodes are arranged in a planar surface that is arranged in parallel to the irradiation zone. In a preferred embodiment, light-emitting diodes arranged in parallel to the irradiation zone are situated on two opposite sides of the preferably likewise planar irradiation zone. High efficiency on irradiation is achieved by this two-sided irradiation.

The irradiation zone of the device according to the invention comprises one or more channels in which the medium that is to be irradiated is transported through the irradiation zone.

One channel can have, for example, a semicircular, rectangular, trapezoidal or triangular cross section. Preferably it is constructed to be rectangular or semicircular. Particularly preferably it is constructed to be semicircular.

A channel is distinguished by a depth in the range from 10 μm to 2000 μm, particularly preferably in the range from 500 μm to 1000 μm. Such a depth ensures that, even in the case of opaque media, the radiation passes through the medium in the irradiation zone completely.

To avoid a high pressure drop, a channel is between 10 mm and 50 mm wide, particularly preferably between 15 mm and 40 mm wide. The said channel widths additionally have the advantage that they provide a high irradiation area, and so the radiation energy is utilized efficiently.

The combination of volume ratio of the irradiation zone to the device volume in combination with the said dimensions and geometries surprisingly leads to a particularly efficient utilization of the radiation used (light yield).

One or more channels, according to the surface to be illuminated and the desired plant volume, can proceed linearly, in a meander-shaped manner and/or in the form of a plurality of parallel strings. Preference is given to an embodiment in which one or two parallel channels proceed in a meander-shaped manner in the irradiation zone. In this way, simple adaptation to the radiation characteristics of the radiation source can be achieved with a minimum pressure drop.

In the case of a meander-shaped flow guidance, the channels in the deflection points preferably have taperings which effect an increase in the flow velocity at these points and therefore a prevention or at least reduction of deposits. With an appropriate design, the taperings, depending on the flow velocity, can also serve as a detachment edge for gas bubbles which are thereby reduced in size at this point.

The guidance of the channels is preferably matched to the arrangement of the radiation source in such a manner that the emission cone of the radiation source is optimally utilized at the appropriate distance.

By targeted guidance of media, also the fraction of the more poorly illuminated sites is decreased—in particular when a radiation source comprising a plurality of light-emitting diodes is used in which, owing to the design, interstices are formed between two adjacent light-emitting diodes, in which interstices the radiation intensity is decreased. Therefore, the ridges between the channels are preferably constructed in such a manner that as little medium as possible is guided in poorly illuminated or non-illuminated interstices.

Alternative radiation sources such as, in particular, excimer lamps which can be produced in many different dimensions, or metal vapour lamps can likewise be used, but, owing to the emission characteristics which frequently additionally require a reflector, do not exhibit energy utilization quite as high as light-emitting diodes.

In a preferred embodiment, the irradiation zone comprises a planar reaction zone plate in which the channels are incorporated using microtechnical assembly processes. By a covering, the channels are sealed in the direction of the radiation source. The covering is adapted in such a manner that it has sufficient transparency for the radiation used.

The reaction plate can be fabricated from metal such as, e.g. stainless steel, Hastelloy, titanium, Monel or plastics such as, e.g., perfluorinated polymer compounds (PFTE), glass or graphite. The reaction zone plate can be generated from suitable semimanufactured products using machining processes, from plastics, or by injection-moulding or embossing techniques.

It is likewise conceivable to implement the channels by using one or more spacers, for example made of metal or plastic, which are arranged between two covers.

As materials for the transparent covering, depending on the requirement of the transmission for irradiation used, quartz glass, glass or transparent plastic such as Perspex can be used.

Also, heating/cooling channels are preferably incorporated into the irradiation zone, to which channels a thermal fluid can be connected for thermal control. In addition, at least one temperature sensor is present which makes possible temperature control of the irradiated medium.

The device according to the invention further comprises means for transporting through the irradiation zone the medium that is to be irradiated. By means of this transport means, the flow velocity and thereby the dwell time of the medium in the irradiation zone can be set in a targeted manner. In combination with a changeable radiation intensity, therefore the radiation dose of the medium that is to be irradiated can be set exactly. As means for transport, for example, a pump (peristaltic pump, gear pump, diaphragm pump, piston pump or centrifugal pump) can be used.

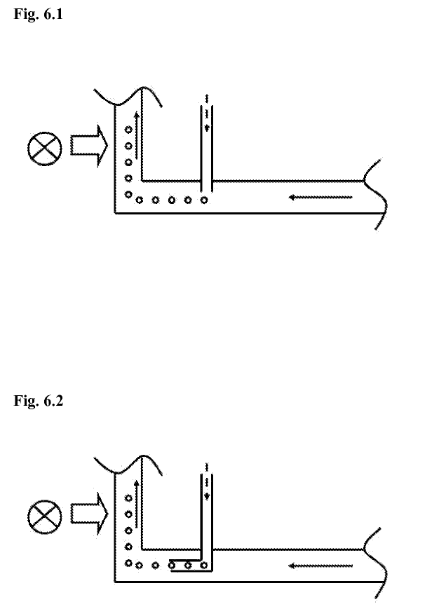

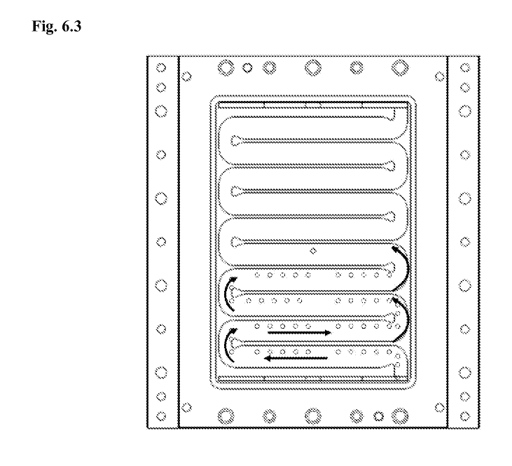

In a particularly preferred embodiment, the device according to the invention is designed in such a manner that the entry region of the irradiation zone is below the exit region of the irradiation zone, with respect to the direction of gravity. In consequence, the medium must be transported through the irradiation zone against the force of gravity. This embodiment is advantageous, in particular, when, in addition a gas feed in the entry region proceeds in the irradiation zone, since in this case an extremely advantageous mixing of the medium with the gas that is fed in occurs. Such an embodiment is depicted by way of example in FIGS. 6.1 to 6.3.

It has now surprisingly been found that a photoreactor having channel dimensions in the depth range from 10 to 2000 μm and for a width in the range from 5 mm to 200 mm, having a gas feed in the media feed mounted against the direction of gravity, wherein finely divided gas bubbles are generated in the irradiation zone, in combination with peripherals such as sensors, pump appliance, gas separation appliance, which contains not more than half of the total volume, is particularly suitable for carrying out photochemical processes with gases and liquids and also photobiological processes such as culturing phototropic microorganisms.

In one such embodiment, the device according to the invention uses the gas necessary for carrying out the photochemical or photobiological process in a finely divided form in a microstructured irradiation zone of the photoreactor in order, firstly, not to impair too greatly rapid resupply of the gas to the liquid phase, and secondly not to impair too greatly the irradiatable surface of the liquid phase.

In rapid photochemical reactions, owing to the presence of the gas having a high phase boundary area, the concentration of the dissolved gas and products thereof as reaction partners are kept high. In photobiological applications, the gas bubbles ensure additional mixing and a decrease of deposits in the irradiation zone.

The media feed and outlet mounted against the direction of gravity make possible advantageous filling, deaeration of the irradiation zone and also a narrow dwell time distribution of the medium that is to be irradiated.

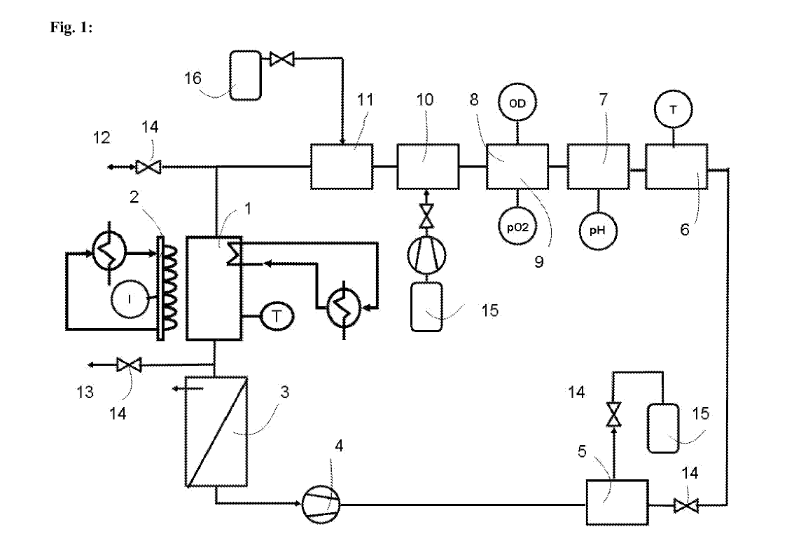

An exemplary plant description of such an embodiment will follow hereinafter with reference to FIG. 1, wherein the figures are to be understood only as typical values and are not a restriction.

The total volume of the medium in this case was 35 ml, the reactor volume, in total, 20 ml, the total volume of the vessel was 10 ml. The channels were 800 μm deep and 20 mm wide. The photoreactor had heating/cooling channels and a temperature sensor and was heated/cooled using circulated water from 23° C. The flow rate during operation is 10 ml/min, the gas introduction was controlled automatically depending on the pH, via control valves. The pressure and temperature sensors used were volume-minimized The optical density was determined in a flow cell made of quartz glass via a fibre-optic microspectrometer. The pump used was a centrifugal pump having a small internal volume.

Preferably, the device according to the invention further comprises a detector for measuring the radiation intensity. The detector determines either the radiation intensity directly or a parameter connected thereto. A suitable detector is, for example, a photodiode or a phototransistor which convert incident electromagnetic radiation into an electrical signal.

The detector in this case must be mounted in such a manner that detection as representative as possible of the radiation is ensured. Preferably, it is therefore mounted laterally on the transparent covering in such a manner that scattered light which is propagated in the covering is detected, particularly preferably, however, directly adjacently to each individual radiation source or adjacently to groups of radiation sources. In the case of light-emitting diodes, for example, in each individual light-emitting diode housing, a photodiode can be accommodated. This ensures optimum monitoring and recording of the radiation intensity. In addition, measurement of the instantaneous current flux by each light-emitting diode or groups of light-emitting diodes permit monitoring of the radiation intensity, provided that variations of the radiation intensity owing to ageing processes or other changes in the light-emitting diodes are known and taken into account.

In addition, the device can comprise various sensors for monitoring pH, ion concentration, pressure and temperature, and also for the optical detection of light absorption and/or light scattering, and a process control appliance for automation.

The device according to the invention may be used for a plurality of photochemical processes. It can be operated continuously, in batch method or semi-batch method.

Surprisingly, it has been found that the device according to the invention can be used for culturing photosynthesizing cells and microorganisms. The present invention therefore also relates to the use of the device according to the invention for culturing photosynthesizing cells and/or microorganisms.

In particular, it was surprising that culturing photosynthesizing cells and microorganisms can be carried out in the device according to the invention reproducibly on a scale under conditions which are very similar to the conditions prevailing in large industrial plants. Applicability of the results from the device according to the invention to a larger production scale is possible thereby. In this case, for example, light input, temperature, flow velocity, gas exchange and nutrient supply can be changed on a small scale in order to identify optimum culture conditions without wasting large amounts of materials. The device according to the invention therefore permits effective studies of optimum growth conditions, the stabilization of all important parameters such as temperature, pH, gas exchange, it allows high cell count densities and thus a high biomass yield and it uses radiation units which are variable in wavelength and intensity. By means of the plant design, scalability of the results is achieved.

In particular, the use according to the invention of the device for culturing photosynthesizing cells and microorganisms makes it possible to generate starter cultures for large-scale plants, wherein these starter cultures are better adapted to the conditions of the large-scale plants than cultures from, e.g., shaken flasks. The starter cultures serve, if necessary, after stepwise multiplication, for inoculating large-scale plants.

A device according to the invention which is used as photobioreactor for culturing photosynthesizing cells and/or microorganisms comprises at least one irradiation zone, a source of electromagnetic radiation and means for transporting a medium through the device.

For the use according to the invention, the photobioreactor is preferably operated continuously in a cycle, that is to say the medium that is to be irradiated containing the cells and/or microorganisms flows through the photobioreactor continuously in a cycle.

The irradiation zone is preferably characterized in that the volume of the irradiation zone corresponds at least to 0.5 times the device volume. Therefore the cells and/or microorganisms in the case of continuous cyclic operation dwell for at least the same time in the irradiated zones as in the non-irradiated zones.

As radiation source, preferably light-emitting diodes are used which are preferably arranged in one or two planar surfaces and are orientated in parallel to the irradiation zone.

Preferably, for culturing photosynthesizing cells and/or microorganisms, light-emitting diodes are used which emit light in the range of the visible blue and/or red range of the spectrum.

The number of channels in the irradiation zone is preferably one or two. In the case of more than two channels, owing to the deposits frequently observed in cell suspensions, differing volumetric flow rates and therefore differing irradiation times in individual channels can occur.

The means for transporting the medium through the photobioreactor should have as low a shear rate as possible, since otherwise a load is exerted on the cells or microorganisms which can lead to a reduction in productivity owing to stressed and/or dead cells or microorganisms, and in product quality due to lysis products. Preferably, peristaltic pumps, piston pumps, gear pumps or centrifugal pumps are used, wherein these preferably have low pump volumes and low speeds of rotation. Studies have found that the result of culturing can be just as favourably influenced by automatic control of the speed of rotation of the pump in the course of culture, as by the specific adjustment of the radiation intensity. The optimum course of a culture is maintained reproducibly by the automation technique used after an experimental phase for determining the optimum parameters.

Preferably, the device has a gas feed and gas takeoff in order to supply the cells or microorganisms with gaseous nutrients and to dispose of gaseous metabolic products. Preferably, the gas feed proceeds directly in the inlet of the irradiation zone using a nozzle having a diameter in the range from 10 μm to 1000 μm, in such a manner that small gas bubbles having diameters below 1 mm migrate through the irradiation zone of the photobioreactor. In this case a high surface area and therefore an effective mass transport between gas and liquid is made possible, in order, for example, to permit the introduction of as much CO₂ as possible into a cell suspension and to permit the discharge of the oxygen formed in the photosynthesis.

The arrangement with gas injection can also be used for carrying out photochemical reactions employing gases such as, e.g., photohalogenations or photooxidations.

The photobioreactor used according to the invention further comprises, preferably, sensors for pH, oxygen, temperature, pressure and optical monitoring, pumps and valve technology, piping, appliances for data recording and process automation. It may be noted that the plant section which is, inter alia, not irradiated, has a liquid volume as low as possible.

The invention will be described in more detail hereinafter with respect to examples, without restricting it thereto.

In the drawings:

FIG. 1 shows a process diagram of a preferred embodiment of the device according to the invention for culturing photosynthesizing cells and/or microorganisms

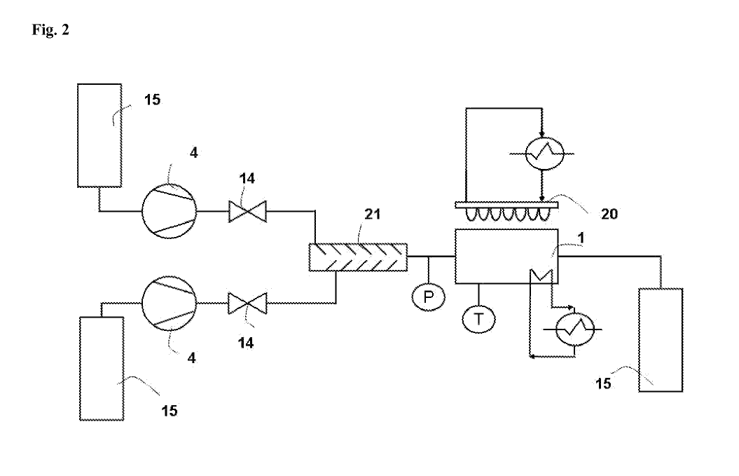

FIG. 2 shows a process diagram of a preferred embodiment of the device according to the invention of a typical plant for carrying out photochemical processes using a micromixer for mixing reactive species

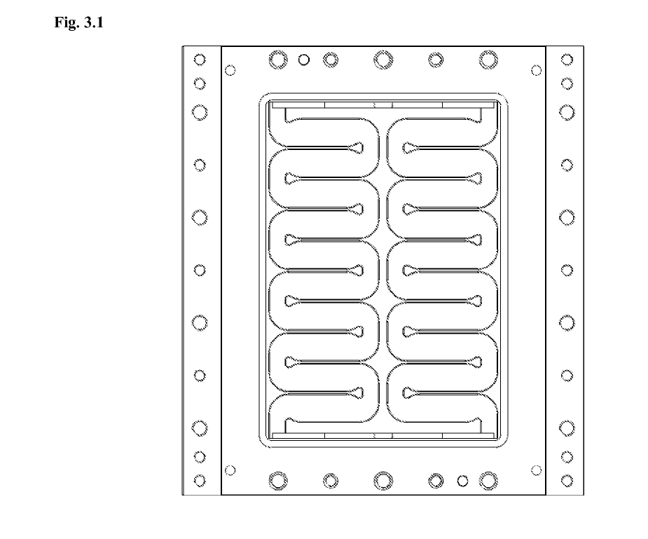

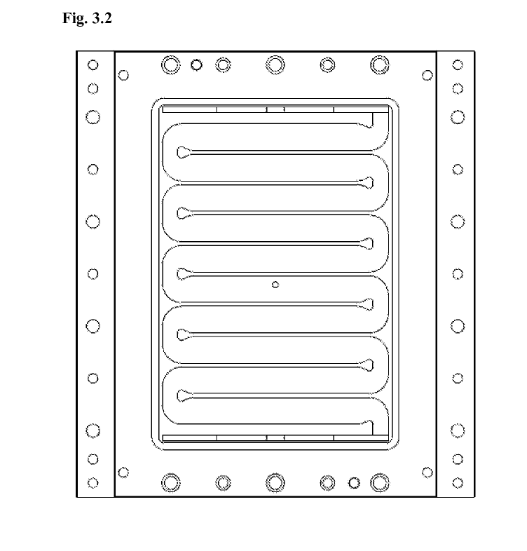

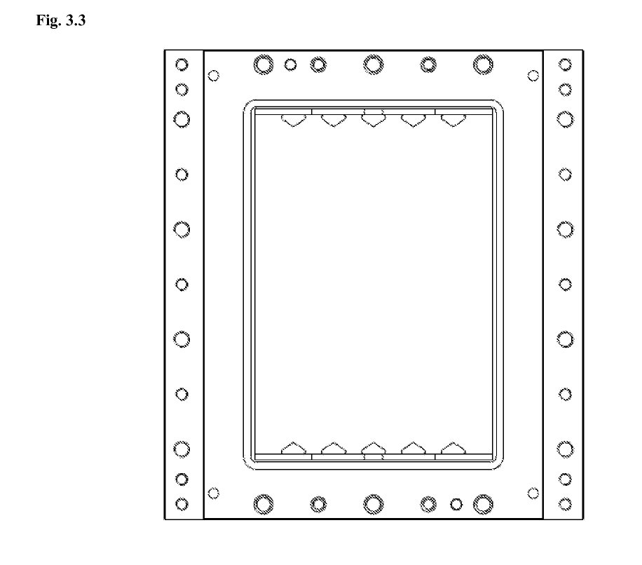

FIG. 3.1 shows an exemplary embodiment of the channel design: two meander-shaped channels running in parallel

FIG. 3.2 shows an exemplary embodiment of the channel design: a single meander-shaped channel

FIG. 3.3 shows an exemplary embodiment of the channel design: a gap-shaped surface having a liquid distribution structure through which flow passes from the bottom

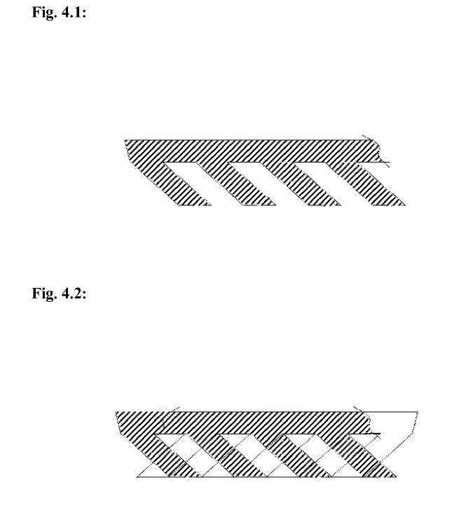

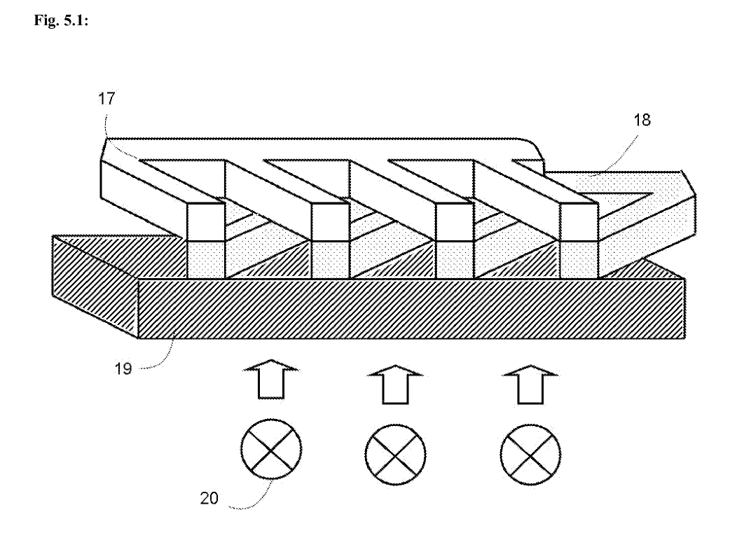

FIG. 4.1 shows a structured metal sheet or structured plate for generating thin irradiated layers

FIG. 4.2 shows an arrangement of two structured metal sheets or plates which are stacked opposite to one another

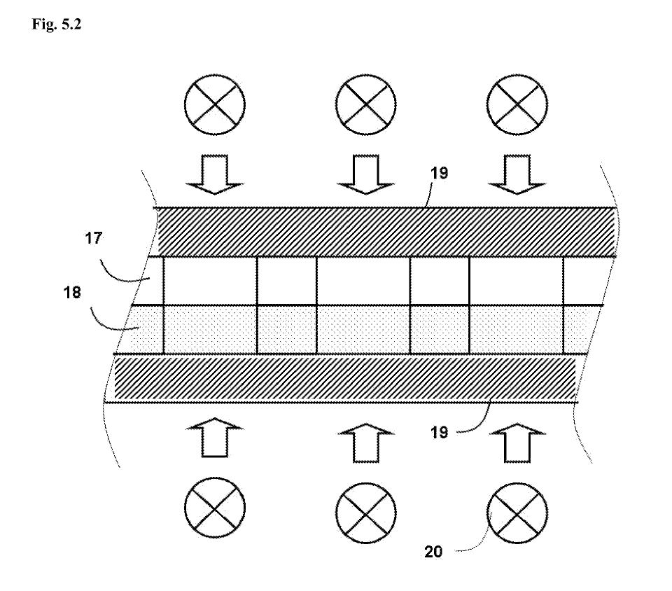

FIG. 5.1 shows diagrammatic representations of arrangements of structured stacked plates having a transparent covering and radiation sources, which are irradiated from one side

FIG. 5.2 shows diagrammatic representations of arrangements of the structured stacked plates having a transparent covering and radiation sources, which are irradiated from two sides

FIG. 6.1 shows a diagrammatic representation of a part of the device according to the invention having an irradiation zone and a gas inlet which is mounted in the intake region of the irradiation zone, in cross section from the side

FIG. 6.2 shows a diagrammatic representation of a part of the device according to the invention having an irradiation zone and a gas inlet which is mounted in the intake region of the irradiation zone, in cross section from the side

FIG. 6.3 shows a diagrammatic representation of a channel running in a meander-shaped manner having taperings at the deflection points, in which gas bubbles migrate through the irradiation zone together with the flow of the medium (indicated by the arrows).

In FIG. 1, a preferred embodiment of the device according to the invention for culturing photosynthesizing cells and/or microorganisms is shown diagrammatically. The device comprises an irradiation zone (1) and attached peripherals having pump and valve technology, various sensors for pH (7), pressure and temperature monitoring (6) and for the optical detection of light absorption (8) and light scattering, heating/cooling appliances, piping and a process control appliance for automation. In addition to the energy transfer, the mass transfer plays an important role, in particular the CO₂ introduction, the oxygen discharge, the nutrient supply and optionally separating off toxic metabolites.

Gas input is achieved via fine nozzles in the entry to the irradiation zone (11), and also FIG. 6.1, 6.2. Fine gas bubbles having diameters less than 1 mm are generated at the entrance to the irradiation zone and migrate through the irradiation zone. In this case, matching of the flow velocity of liquid and gas phases occurs in order to ensure transport of the gas bubbles through the device, in particular through the irradiation chamber and to decrease coalescence. Coalescence leads to enlarged gas bubbles, to decreased surface areas between gas and liquid and therefore to impaired mass transfer.

Downstream of the outlet of the irradiation zone there is situated an equilibration vessel (3) which serves for separating off gas and liquid. The gas separated off here can also be used for reinjection, provided that the oxygen content is not too high.

The valve technology of the injection of the CO₂-containing gas forms, together with the pH sensor and the appliance for data recording and control, a control unit using which the pH is kept constant in the suspension that is circulated by pumping. A further injection site serves for optional addition of nutrient solution, depending on the growth phase or recorded parameters. An optical flow cell serves for detecting optical parameters such as absorption, light scattering properties or fluorescence. It has proved to be advantageous if all of the components are connected to one another with lowest possible volume and friction-fitting or positive-lock connections. The components can readily be removed thereby, changed or used at another point in the process diagram which, in the context of studies or optimization tasks, offers a time advantage in conversion or cleaning work.

Further sensors, in addition to temperature, which is preferably controlled in the reactor chamber, in the buffer vessel and the pump, also detect the oxygen partial pressure electrochemically or fibre-optically with the aid of what is termed luminescence optodes. An optical detector detects the optical density which correlates with the biomass. Coupling in a fibre-optic spectrometer is also expedient, and so spectrally resolved measurement of absorption and light scattering is possible. As a result, firstly, the growth can be pursued on-line, but also, using absorption-spectroscopic measurements, the colorant content can be examined.

It has proved to be advantageous to harvest the cultures when a certain cell count density or colorant concentration is achieved and preferably has been indicated by the sensors used.

FIG. 2 shows the process diagram of a preferred embodiment of the device according to the invention for carrying out photochemical reactions, wherein a micromixer for metering liquids such as, e.g., reaction components, can be connected upstream of the actual photochemical reaction in the irradiation zone.

The individual functionalities of the devices in FIGS. 1 and 2 are preferably constructed in a modular manner, such that modifications of the plant diagram are readily and rapidly possible. Particular preference is given to the use of a frictional fit or positive-fit connection without piping or other connection technology between the individual modules in order to minimize the plant volume.

A typical embodiment of the channel design in the irradiation zone is shown in FIG. 3.1. The medium is conducted in two channels in a meander-shaped manner upstream of the radiation source, wherein, when a plurality of light-emitting diodes are used, the position and the distance of these are selected in such a manner that the channels are completely illuminated. The irradiation zone is preferably erected vertically, in such a manner that flow passes through the channels from bottom to top against the force of gravity. Further expedient practical channel designs are shown in FIGS. 3.2 and 3.3. There, either only one individual channel is used for liquid guidance (FIG. 3.2) or else a single large gap having suitable liquid distribution at the entrance to the irradiation zone is used (FIG. 3.3).

In a preferred embodiment, flow inserts are used in order to generate the thin irradiated layer. The flow inserts preferably consist of structured metal sheets or plates which are inserted stackwise into the channel. As a result, no fine structures need to be incorporated in the irradiation zone (reactor zone plate) itself The arrangement of channel structures and flow inserts is therefore demountable, can be organized variably, and may be cleaned readily.

In FIGS. 6.1 and 6.2, a part of the device according to the invention is shown. This part comprises a gas inlet in the intake region of the irradiation zone. FIGS. 6.1 and 6.2 show various embodiments of the gas inlet. Via a nozzle, small gas bubbles are introduced into the medium (shown by the dashed arrow and the small circle) which are entrained by the medium by the flow (shown by the thin continuous arrow). The medium flows through the preferably vertically orientated irradiation zone preferably from bottom to top. The irradiation is indicated by the thick arrow. The radiation source is shown, as is generally customary, by a circle having two crossing lines.

FIG. 6.3 shows the arrangement of FIGS. 6.1 and 6.2 in a plan view. The irradiation zone is irradiated from the line of sight of the viewer. It comprises a channel running in a meander-shaped manner through the irradiation zone. Gas bubbles which are introduced in the intake region of the irradiation zone migrate through the irradiation zone together with the medium (shown by the arrows).

### Reference Signs

1 Irradiation zone with liquid guidance, thermostatting liquid, temperature sensor
2 Irradiation unit with thermostatting liquid and intensity measurement
3 Equilibration vessel with gas separation
4 Pump
5 Filtration unit
6 Temperature sensor
7 pH sensor
8 Optical sensor for transmission
9 Oxygen sensor
10 Nutrient addition
11 Gas injection
12 Take-off and filling opening
13 Drainage opening
14 Shut-off valve
15 Liquid vessel
16 Gas vessel
17 Structured metal sheet or plate
18 Structured metal sheet or plate
19 Covering
20 Radiation source
21 Micromixer

1. Device for carrying out photochemical and photobiotechnological processes, comprising at least a microstructured irradiation zone, a source of electromagnetic radiation and means for transporting a medium through the device, wherein the volume of the irradiation zone corresponds to at least 0.5 times the device volume, and the irradiation zone comprises one or more channels which pass through the irradiation zone in a linear or meander-shaped manner and are constructed so as to be rectangular or semicircular in cross section and have a depth in the range from 10 μm to 2000 μm.

2. Device according to claim 1, wherein the channels have a depth in the range from 500 μm to 1000 μm.

3. Device according to claim 1 or 2 wherein the channels have a width in the range from 10 mm to 50 mm.

4. Device according to claim 3, wherein the channels have a width in the range from 15 mm to 40 mm.

5. Device according to claim 1, wherein the microstructured irradiation zone is equipped with a media inlet that is provided mounted at the bottom in the direction of gravity and a media outlet that is mounted at the top in the direction of gravity.

6. Device according to claim 1, wherein the media inlet that is provided mounted at the bottom in the direction of gravity is provided with a gas feed.

7. Device according to claim 1, wherein the channels have taperings at their deflection points.

8. Device according to claim 1, wherein the radiation source is an arrangement of light-emitting diodes in a planar surface that is arranged in parallel to the irradiation zone.

9. Device according to claim 1, wherein the irradiation zone is mounted between two planar arrangements of light-emitting diodes, and the arrangements of light-emitting diodes and the irradiation zone are orientated in parallel to one another.

10. Device according to claim 1, wherein the means for transporting a medium through the device is a peristaltic pump, piston pump, gear pump, diaphragm pump or centrifugal pump.

11. Device according to claim 1, wherein, for development of one or more thin layers of the medium in the irradiation chamber, layers of structured metal sheets or plates are introduced.

12. Method for culturing photosynthesizing cells or microorganisms which comprises culturing said culturing photosynthesizing cells or microorganisms in the device of claim 1.

13. Method according to claim 12, wherein, in the irradiation zone, gas bubbles having a diameter of less than 1 mm are generated, in such a manner that the gas bubbles migrate through the irradiation zone together with the medium.

14. Method according to either of claim 12 or 13, wherein the irradiation zone is orientated vertically, and the medium flows through the irradiation zone from bottom to top against the direction of gravity.

15. Method according to claim 12 for generating starter cultures for a plant.
